(19) United States
(12) Patent Application Publication
(10) Pub. No.: US 2020/0299645 A1
(43) Pub. Date: Sep. 24, 2020

(54) METHOD FOR PREPARING ANTIGEN-SPECIFIC REGULATORY T CELLS

(71) Applicant: REGCELL CO., LTD., Kyoto (JP)

(72) Inventors: Hiroshi KAWAMOTO, Kyoto (JP); Shimon SAKAGUCHI, Kyoto (JP); Kyoko MASUDA, Kyoto (JP); Keiji HIROTA, Kyoto (JP); Junji UEHORI, Kyoto (JP)

(21) Appl. No.: 16/473,394

(22) PCT Filed: Dec. 27, 2017
(86) PCT No.: PCT/JP2017/047014

§ 371 Date: Jun. 1, 2020

(30) Foreign Application Priority Data
Dec. 27, 2016 (JP) 2016-254095

(51) Int. Cl. C12N 5/0783

(52) U.S. Cl. CPC C12N 5/0637; C12N 2506/45; C12N 2501/999; C12N 2502/1121; C12N 2506/11

(57) ABSTRACT

Provided is a method for producing regulatory T cells for inducing immune tolerance in a subject. The method comprises the step of co-culturing regulatory T cells obtained from the subject with dendritic cells derived from iPS cells.

### TECHNICAL FIELD

The present application relates to a method for inducing antigen-specific regulatory T cells that are used for inducing immune tolerance.

### ART RELATED

Regulatory T cells were discovered as a cell population in the peripheral CD4-positive T cells that inhibit autoimmune reactions. At present, the regulatory T cells are known to inhibit not only autoimmune reactions but also tumor immunity, transplantation immunity, allergic reactions and immune reactions against infection. For example, Non Patent Literature 1 describes that antigen-specific regulatory T cells can specifically suppress rejection of skin transplants.

Non Patent Literature 2 describes an idea that antigen-specific regulatory T cells are induced in vitro and used for suppressing rejection of organ transplants. Currently, administration to a patient of antigen-specific regulatory T cells that are derived from the patient and selectively amplified in vitro has been examined for treating autoimmune diseases, organ transplants rejection, graft-versus-host disease (GVHD), allergic disease and the like.

For amplifying antigen-specific regulatory T cells in vitro, the regulatory T cells derived from the patient may be co-cultured with dendritic cells derived from monocytes of the same patient or with dendritic cells derived from monocytes of the transplant donor. However, it is difficult to prepare a sufficient amount of monocytes from the patient or the donor, and the burden on the patient could be large.

Clinical studies for inducing immune tolerance in a patient by using regulatory T cells in living donor liver transplantations have been conducted (Non Patent Literature 3). The cells used in this method were not proper regulatory T cells but anergy T cells obtained by co-culturing T cells and dendritic cells with suppressing the sub-stimulating factor and in the document, thus obtained T cells were referred to as “regulatory T cells” (Patent Literature 1).

Methods for producing dendritic cells from pluripotent stem cells such as ES cells and iPS cells have been known (for example, Patent Literature 2).

### CITATION LIST

### Patent Literature

[Patent Literature 1] Japanese Patent Laid-Open No. 2016-520081
[Patent Literature 2] National Publication of International Patent Application No. 2014-506447

### Non Patent Literature

[Non Patent Literature 1] Nagahama K., Sakaguchi S., et al., Differential control of allo-antigen-specific regulatory T cells and effector T cells by anti-CD4 and other agents in establishing transplantation tolerance, Int Immunol. 21:379, 2009
[Non Patent Literature 2] Takasato F., Yoshimura A., et al., Prevention of allogeneic cardiac graft rejection by transfer of ex vivo expanded antigen-specific regulatory T-cells, PLoS One. 9(2): e87722, 2014
[Non Patent Literature 3] “Tolerance induction by a regulatory T cell-based cell therapy in living donor liver transplantation”, UMIN ID: UMIN000015789 (https://upload.umin.ac.jp/cgi-open-bin/ctr/ctr_view.cgi?recptno=R000018372)

The disclosures of the above patent and non-patent literatures are herein incorporated by reference.

### SUMMARY OF INVENTION

An object of the present application is to provide a method for inducing antigen-specific regulatory T cells outside the body. Another object of the present application is to provide a method for inducing immune tolerance in a subject by using thus induced antigen-specific regulatory T cells.

### Solution to Problem

The present application provides a method for producing regulatory T cells for inducing immune tolerance in a subject, comprising the step of co-culturing regulatory T cells obtained from the subject with dendritic cells derived from iPS cells. The regulatory T cells obtained by the method of the present application are useful for the treatment of an autoimmune disease, organ transplant rejection, graft-versus-host disease (GVHD), allergic disease and the like in the subject.

The first aspect of the present application provides a method for inducing antigen-specific regulatory T cells for inducing immune tolerance in a subject, comprising the steps of:
providing iPS cells established from a somatic cell of a somatic cell donor having HLA class II molecules that match with those of the subject to a certain extent or more;
inducing dendritic cells from the iPS cells;
sensitizing the dendritic cells with an antigen to which the immune tolerance is to be induced; and
co-culturing regulatory T cells obtained from the subject with the antigen-presenting dendritic cells.

The second aspect of the present application provides a method for inducing antigen-specific regulatory T cells for inducing immune tolerance in a transplant recipient to a tissue of a transplant donor, comprising the steps of:
providing iPS cells established from a somatic cell derived from a somatic cell donor having HLA class II molecules that match with those of the transplant donor to a certain extent or more;
inducing dendritic cells from the iPS cells; and
co-culturing regulatory T cells obtained from the transplant recipient with the induced dendritic cells.

The third aspect of the present application provides a method for inducing antigen-specific regulatory T cells for inducing immune tolerance in a transplant recipient to a tissue of a transplant donor, comprising the steps of:
providing iPS cells established from a somatic cell derived from a somatic cell donor having HLA class II molecules that match with those of the transplant recipient to a certain extent or more;
inducing dendritic cells from the iPS cells;
sensitizing the dendritic cells with an antigen derived from the transplant donor; and
co-culturing regulatory T cells obtained from the transplant recipient with the induced antigen-presenting dendritic cells.

The fourth aspect of the present application provides a method for inducing antigen-specific regulatory T cells for inducing immune tolerance in a tissue of a transplant donor to a transplant recipient that tolerates the immune reaction caused by the T cells of the transplant donor, comprising the steps of:
preparing iPS cells established from a somatic cell derived from a somatic cell donor having HLA class II molecules that match with those of the transplant recipient to a certain extent or more;
inducing dendritic cells from the iPS cells; and
co-culturing regulatory T cells obtained from the transplant donor with the induced dendritic cells.

The fifth aspect of the present application provides a method for inducing antigen-specific regulatory T cells for inducing immune tolerance in a tissue of a transplant donor against a transplant recipient that tolerates the immune reaction caused by the T cells of the transplant donor, comprising the steps of:
providing iPS cells established from a somatic cell derived from a somatic cell donor having HLA class II molecules that match with those of the transplant donor to a certain extent or more;
inducing dendritic cells from the iPS cells;
sensitizing the dendritic cells with an antigen derived from the transplant recipient; and co-culturing regulatory T cells obtained from the transplant donor with the antigen-presenting dendritic cells.

### Effects of the Invention

In this method, dendritic cells derived from iPS cells are used and therefore, a large amount of the antigen presenting dendritic cells can be provided, and hence, antigen-specific regulatory T cells can be stably produced in a large amount.

### BRIEF DESCRIPTION OF DRAWINGS

FIG. 1 is a conceptual diagram illustrating example 1.

FIG. 2 shows the ratio of the cell proliferation each group performed in example 1.

FIG. 3 shows the result of example 2. Dendritic cells derived from HLA haplotype-homozygous iPS cells were co-cultured with regulatory T cells obtained from a healthy volunteer whose HLA haplotypes do not match any of the HLA haplotypes of the iPS cells. As a result, regulatory T cells expressing Foxp3 were proliferated.

### DESCRIPTION OF EMBODIMENTS

In the specification and claims, the term “somatic cell donor” refers to a donor who provides somatic cells to be used for establishing iPS cells.

In the first aspect of the present application, the somatic cell donor needs to have HLA class II molecules that match with those of the subject to a certain extent or more. By the expression “HLA class II molecules match between two samples to a certain extent or more”, it is meant that among the three HLA class II molecules of DR, DP and DQ, molecules capable of presenting the target antigen match between the two samples. iPS cells maintain the HLA molecules of the somatic cell from which the iPS cells are induced. When the iPS cells are differentiated into dendritic cells, the dendritic cells maintain the original HLA molecules. A T cell recognizes only one HLA molecule. iPS cells induced from a somatic cell of a somatic cell donor who has at least one HLA class II molecules that matches with that of the patient may be differentiated into dendritic cells, and the dendritic cells may be used to proliferate the regulatory T cells that are reactive to an antigen that binds to the HLA class II molecule. If only one HLA class II molecule among the three HLA class II molecules of the somatic cell donor matches with that of the subject while the others mismatch, regulatory T cells reactive to the other HLA molecule, i.e. alloreactive regulatory T cells may be grown, and hence the efficiency for obtaining necessary cells may be lowered. Accordingly, all the three HLA class II molecules desirably match between the somatic cell donor and the subject. The somatic cell donor may be the subject itself in which the immune tolerance is to be induced.

The iPS cell stock project is now being strongly promoted in Japan. Under this project, a highly versatile iPS cell bank is created with donors having HLA haplotypes that are frequently found in Japanese people in homozygous. The HLA haplotype-homozygous iPS cells (haplotype-homo iPS cells) in the stock are distributed to research institutions as well as medical institutions so that the cells are widely used in regenerating therapies. In the first aspect of the present application, if a patient is heterozygous for the HLA haplotypes, iPS cells obtained from a donor having one of the HLA haplotypes of the patient homozygously can be used. Suitable iPS cells can be selected from the iPS cells stocked in the iPS cell stock project or the other iPS cell banks on the basis of the donor's HLA and the other information that are stored in connection with the iPS cells.

An antigen to be presented by the dendritic cells is not particularly limited as long as immune tolerance to the antigen is desired to be induced, and examples include antigens that cause autoimmune diseases and allergic diseases. Examples of antigens include, but are not limited to, protein antigens, peptide antigens, non-peptide antigens, for example, phospholipids and complex carbohydrates (for example, bacterial membrane components such as mycolic acid and lipoarabinomannan).

The first aspect of the present application is a method for preparing regulatory T cells for inducing immune tolerance to a specific antigen for treating an autoimmune disease, an allergic disease or the like. Dendritic cells presenting an antigen together with the HLA class II molecules same as those in a subject into which the immune tolerance is induced are prepared, and regulatory T cells derived from the subject are cultured with the antigen presenting dendritic cells so that the antigen-specific regulatory T cells are selectively amplified.

In the second and third aspects of the present application, antigen-specific regulatory T cells for inhibiting the attack by the transplant-recipient's immune system targeting a graft, mainly the graft in an allo-transplantation are prepared.

In the second aspect, the reaction directly caused by the recipient's T cells against an HLA molecule that is expressed in the graft but not in the recipient is inhibited. Regulatory T cells alloreactive to the graft are prepared. It is known that many of organ transplant rejections are caused through the direct reaction of the recipient's T cells to an HLA molecule that is not expressed in the recipient but is expressed in the graft. In the normal state, somatic cells express HLA class I molecules only. Once inflammation occurs and interferon or the like is produced in the vicinity, HLA class II molecules start to be expressed in the T cells. When the cells reactive to the HLA class II molecule expressed in the graft are amplified from the regulatory T cells of the recipient and the amplified cells are administered, the administered regulatory T cells expect to inhibit the rejection against the graft.

In the second aspect, the somatic cell donor needs to have HLA class II molecules that match with those of the transplant donor to a certain extent or more. In the second aspect, the term “have HLA class II molecules that match with those of the transplant donor to a certain extent or more” means that when the HLA class II molecules of the transplant donor do not match with those of the transplant recipient, the somatic cell donor has at least one of the HLA class II molecules of the transplant donor out of the mismatching molecules. Preferably, the somatic cell donor has homozygous or heterozygous HLA class II haplotypes that match completely with the HLA class II molecules of the transplant donor. The somatic cell donor may be the same person as the transplant donor. When tissues or cells differentiated from iPS cells are transplanted, the same iPS cells may be used for preparing the dendritic cells.

In the second aspect, immune tolerance targeting the HLA class II molecules of the transplant donor is induced.

The third aspect of the present application is a method for preparing regulatory T cells that inhibit rejection caused by the recipient's immune system targeting a graft, i.e. a foreign matter, through the antigen presentation to its own dendritic cells. In the third aspect, in the same manner as in the first aspect, the somatic cell donor needs to have HLA class. II molecules that match with those of the transplant recipient, that is, the subject to be treated, to a certain extent or more. In the third aspect, the expression “have HLA class II molecules that match to a certain extent or more” means the same as in the first aspect. Examples of an “antigen derived from a graft” include an HLA and a minor histocompatibility antigen of the transplant donor when the HLA molecules do not match between the transplant donor and the recipient.

The fourth and fifth aspects of the present application are methods for inducing regulatory T cells useful for preventing or treating GVHD, mainly after bone marrow transplantation.

The fourth aspect is a method for inducing regulatory T cells derived from a transplant donor and alloreactive to a transplant recipient (allo), so as to inhibit the reaction directly caused by the donor's T cells contained in the graft targeting the HLA molecules expressed in the transplant recipient that mismatch with the HLA molecules expressed in the graft. In the fourth aspect, the somatic cell donor needs to have HLA class II molecules that match with those of the transplant recipient, that is, the subject to be treated, to a certain extent or more. In the fourth aspect, the expression the somatic cell donor “having HLA class II molecules that match with the transplant recipient to a certain extent or more” means that when the HLA class II molecules of the transplant recipient mismatch with those of the transplant donor, the somatic cell donor has at least one HLA class II molecules of the transplant recipient out of the mismatching molecules. Preferably, the somatic cell donor has homozygous or heterozygous HLA class II haplotypes that match completely with the HLA class II molecules of the transplant recipient. The somatic cell donor may be the same person as the transplant recipient.

The fifth aspect is a method for preparing regulatory T cells that inhibit a GVHD caused by immune cells contained in the graft targeting the recipient, i.e. a foreign matter, through antigen presentation to the dendritic cells derived from the graft. In the fifth aspect, the somatic cell donor has HLA class II molecules that match with those of the transplant donor to a certain extent or more. In the fifth aspect, the term “have HLA class II molecules matched to a certain extent or more” means the same as in the first aspect.

In the fifth aspect, examples of an “antigen derived from the transplant recipient” include an HLA molecule and a minor histocompatibility antigen of the transplant recipient in the case that the HLA molecules partly mismatch between the transplant donor and the recipient.

Induced pluripotent stem (iPS) cells can be prepared by introducing specific reprogramming factors to somatic cells. iPS cells are somatic cell-derived artificial stem cells having properties almost equivalent to those of ES cells (K. Takahashi and S. Yamanaka (2006) Cell, 126:663-676; K. Takahashi et al. (2007), Cell, 131:861-872; J. Yu et al. (2007), Science, 318:1917-1920; Nakagawa, M. et al., Nat. Biotechnol. 26:101-106(2008); and WO 2007/069666). The reprogramming factors may be constituted by genes or gene products thereof or non-coding RNAs, which are expressed specifically in ES cells; or genes or gene products thereof, non-coding RNAs or low molecular weight compounds, which play important roles in maintenance of the undifferentiated state of ES cells. Examples of genes included in the reprogramming factors include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tel1, beta-catenin, Lin28b, Sal11, Sal14, Esrrb, Nr5a2, Tbx3 and Glis1, and these reprogramming factors may be used either individually or in combination. Examples of the combination of the reprogramming factors include those described in WO2007/069666; WO2008/118820; WO2009/007852; WO2009/032194; WO2009/058413; WO2009/057831; WO2009/075119; WO2009/079007; WO2009/091659; WO2009/101084; WO2009/101407; WO2009/102983; WO2009/114949; W02009/117439; WO2009/126250; WO2009/126251; WO2009/126655; WO2009/157593; WO2010/009015; WO2010/033906; WO2010/033920; WO2010/042800; WO2010/050626; WO2010/056831; WO2010/068955; WO2010/098419; WO2010/102267; WO2010/111409; WO2010/111422; WO2010/115050; WO2010/124290; WO2010/147395; WO2010/147612; Huangfu D, et al. (2008), Nat. Biotechnol., 26: 795-797; Shi Y, et al. (2008), Cell Stem Cell, 2: 525-528; Eminli S, et al. (2008), Stem Cells. 26:2467-2474; Huangfu D, et al. (2008), Nat Biotechnol. 26: 1269-1275; Shi Y, et al. (2008), Cell Stem Cell, 3, 568-574; Zhao Y, et al. (2008), Cell Stem Cell, 3:475-479; Marson A, (2008), Cell Stem Cell, 3, 132-135; Feng B, et al. (2009), Nat Cell Biol. 1 1:197-203; R. L. Judson et al. (2009), Nat. Biotech., 27:459-461; Lyssiotis C A, et al. (2009), Proc Nati Acad Sci USA. 106:8912-8917; Kim J B, et al. (2009), Nature. 461:649-643; Ichida J K, et al. (2009), Cell Stem Cell. 5:491-503; Heng J C, et al. (2010), Cell Stem Cell. 6: 167-74; Han J, et al. (2010), Nature. 463:1096-100; Mali P, et al. (2010), Stem Cells. 28:713-720, and Maekawa M, et al. (2011), Nature. 474:225-9. The reprogramming factors may be contacted with or introduced into the somatic cells by a known procedure suitable for the form of the factor to be used. Documents cited in this paragraph are herein incorporated by reference.

In the present application, somatic cells used for establishing iPS cells may be any of embryonic somatic cells, neonatal somatic cells and mature healthy or disease somatic cells, and also embrace any of primary culture cells, subculture cells and established cells. Specific examples of the somatic cells include neural stem cells, hematopoietic stem cells, mesenchymal stem cells, tissue stem cells (somatic stem cells) such as dental pulp stem cells, tissue progenitor cells, lymphocytes, epithelial cells, endothelial cells, muscle cells, fibroblasts (such as skin cells), hair cells, liver cells, gastric mucosal cells, intestinal cells, spleen cells, pancreatic cells (such as exocrine pancreas cells), brain cells, lung cells, kidney cells and differentiated cells such as fat cells.

For inducing dendritic cells from iPS cells, any of known procedures for inducing dendritic cells from pluripotent stem cells such as ES cells or iPS cells can be employed. Examples include a method in which an embryoid body is formed for induction in a culture solution supplemented with cytokine (Zhan X., et al., Lancet. 2004, 364, 163-71) and a method in which cells are cultured on heterologous stromal cells (Senju S., et al., Stem Cells, 2007, 25, 2720-9)). As described in Patent Literature 1, a method in which adhesion culture and suspension culture of iPS cells are performed in the absence of feeder cells in a culture medium supplemented with BMP4, VEGF and various hematopoietic factors but not supplemented with serum may be employed. The culture medium is appropriately replaced and the culture in this method. Another example includes a method, employed in an example of the present application, in which iPS cells are differentiated into monocytes by culturing the cells in a culture medium supplemented with GM-CSF and M-CSF, the resultant is then cultured in a culture medium supplemented with 2-mercaptoethanol, GM-CSF and IL-4 to obtain an immature dendritic cells, and the immature dendritic cells are further cultured in the presence of 2-mercaptoethanol, IL-1β, IL-6, TNFα and PGE2 to obtain mature dendritic cells. (Documents cited in this paragraph are herein incorporated by reference.)

The dendritic cells thus induced from the iPS cells are cultured together with regulatory T cells. The regulatory T cells may be those obtained from the subject in which the immune tolerance is to be induced. For transplantation, the regulatory T cells obtained from the transplant recipient are used. The regulatory T cells may be isolated from the peripheral blood of the subject, or the peripheral regulatory T cells may be induced from the peripheral naive CD4-positive T cells. In order to isolate the regulatory T cells from the peripheral blood of the subject, the CD45RA-positive CD25-positive fraction may be taken out using, for example, a cell sorter.

In order to induce the peripheral regulatory T cells from the peripheral naive CD4-positive T cells, any of known methods may be employed, and an example includes a method in which the cells are cultured in the presence of TGFβ.

In the first, third and fifth aspects of the present application, dendritic cells sensitized with an antigen are used. In order to sensitize the dendritic cells with an antigen in vitro, the induced dendritic cells may be brought into contact with the antigen. In the second and fourth aspects of the present application, regulatory T cells specific for an HLA class II molecule of the transplant donor are prepared and used, and sensitization with another antigen is not performed.

In the second and third aspects of the present application, rejection occurring after organ transplantation is mainly dealt with, and immune tolerance targeting an HLA molecule or a minor histocompatibility antigen of the transplant donor is induced.

In the fourth and fifth aspects of the present application, a GVHD occurring after bone marrow transplantation is mainly dealt with, and immune tolerance targeting an HLA molecule or a minor histocompatibility antigen of a transplant recipient is induced.

In the specification and claims, the expression “antigen specific regulatory T cells” represents both regulatory T cells specific for specific HLA class II molecules and regulatory T cells specific for another antigen bound to specific HLA class II molecules.

In the method of the present invention, regulatory T cells obtained from the subject in which immune tolerance is to be induced and the dendritic cells are co-cultured. The culture may be performed in a basal medium for culturing animal cells supplementing with IL-2.

The basal medium for culturing animal cells may be appropriately selected from commercially available culture media, and examples include MEM Zinc Option medium, IMEM Zinc Option medium, IMDM medium, Medium 199, Eagle's Minimum Essential Medium (EMEM), αMEM medium, Dulbecco's modified Eagle's Medium (DMEM), Ham's F12 medium, RPMI 1640 medium, Fischer's medium, and a mixed medium thereof. The basal medium may contain a serum such as fetal bovine serum (FBS) or no serum. If necessary, the culture medium may contain one or more serum replacements such as albumin, transferrin, KnockOut Serum Replacement (KSR) (a serum replacement for use in ES cell culture) (Invitrogen), N2 supplement (Invitrogen), B27 supplement (Invitrogen), fatty acid, insulin, a collagen precursor, a trace element, 2-mercaptoethanol and 3′-thiolglycerol. The medium may contain one or more substances of a lipid, an amino acid, L-glutamine, GlutaMAX (Invitrogen), a non-essential amino acid (NEAA), a vitamin, a growth factor, an antibiotic, an antioxidant, a pyruvic acid, a buffer, an inorganic salt, and equivalents of these.

A concentration of IL-2 in the culture medium may be 1 to 50 U/mL, preferably 5 to 40 U/mL, for example, about 20 U/mL. The medium may further comprise rapamycin. The concentration of rapamycin in the medium, when added, may be 0.5 ng/mL-100 ng/mL, preferably 1-30 ng/mL and for example about 10 ng/mL.

In the specification and claims, the expression “about” is used to include numerical values falling in a range of +20% or ±10%.

A ratio between the dendritic cells and the regulatory T cells (dendritic cells: regulatory T cells) at the beginning of the co-culture is in a range of 1:1 to 20:1, and, for example, is preferably about 10:1. A mixture of the cells is cultured under a conventional culture conditions for animal cells, for example, at 5% CO₂ and 37° C. for about 5 days to about 3 weeks, for example, about 1 to 2 weeks. Incidentally, when cells other than regulatory T cells are unavoidably involved upon isolating the regulatory T cells from the subject even in a small amount, the period of the co-culture may be comparatively short, for example, about 1 week.

After completing the co-culture, the mixed culture of the dendritic cells and the regulatory T cells is dispersed in an appropriate medium to be used for the administration to the subject. Preferably, the dendritic cells are removed from the culture of the regulatory T cells before the administration. For purifying the cells, any known methods may be employed, and separation may be performed using a cell sorter or using microbeads. Examples of the medium for dispersing the cells therein include saline and PBS. The cells may be intravenously administered to the patient. Although it is not restrictive, a dosage may be 10⁷ to 10⁹ cells per individual per administration, and it is administered, for example, intravenously to the patient once or several times.

The regulatory T cells obtained in the first aspect of the present application are useful for treating an autoimmune disease or an allergic disease. The disease to be treated is not particularly limited, and examples include type I diabetes or insulin dependent diabetes, systemic lupus erythematosus, Crohn's disease, cardiomyopathy, hemolytic anemia, fibromyalgia, Graves' disease, ulcerative colitis, vasculitis, multiple sclerosis, myasthenia gravis, myositis, neutropenia, psoriasis, chronic fatigue syndrome, juvenile arthritis, juvenile diabetes, scleroderma, psoriatic arthritis, Sjogren's syndrome, rheumatic fever, chronic rheumatoid arthritis, sarcoidosis, idiopathic thrombocytopenic purpura (ITP), Hashimoto's disease, complex connective tissue disease, interstitial cystitis, pernicious anemia, leukoencephalitis, alopecia areata, ankylosing spondylitis, primary biliary cirrhosis, anti-GBM nephritis, anti-TBM nephritis, antiphospholipid syndrome, polymyalgia rheumatica, polymyositis, autoimmune Addison's disease, chronic active hepatitis, leukoderma vulgaris, autoimmune hyperlipidemia, autoimmune myocarditis, temporal arteritis, autoimmune thyroid disease, axonal and nerve neuropathy, Bechet's disease, bullous pemphigoid, allergic asthma, atopic dermatitis, osteoarthritis, Chagas' disease, uveitis, chronic inflammatory demyelinating polyradiculoneuropathy (CIDP), cicatricial pemphigoid/benign mucous membrane pemphigoid, Cogan syndrome, congenital heart block, Coxsackie myocarditis, demyelinating neuropathy, dermatomyositis, discoid lupus erythematosus, lens antigenic uveitis, polyarteritis nodosa, Dressler syndrome, essential mixed cryoglobulinemia, Evans syndrome, Goodpasture's syndrome, allergic rhinitis, Guillain-Barre syndrome, hypogammaglobulinemia, inclusion body myositis, vesicular bullous dermatitis, Wegener's granulomatosis, Meniere's disease, Lambert-Eaton syndrome, Mooren's ulcer, atypical celiac disease, ocular cicatricial pemphigoid, pemphigus vulgaris, perivenous encephalomyelitis, postpericardiotomy syndrome, scleritis, sperm and testicular autoimmunity, stiff-man syndrome, subacute bacterial endocarditis (SBE), sympathetic ophthalmia, transverse myelitis and necrotic myelopathy, polyglandular autoimmune syndrome type I, polyglandular autoimmune syndrome type II, pernicious anemia, and endometriosis.

The second aspect of the present application is useful for inducing immune tolerance to the graft in any allotransplantation. In the second aspect of the present application, the regulatory T cells can be administered at the same time as the transplantation or can be administered when a rejection occurs. Although not restrictive, a dosage of the regulatory T cells may be 10⁷ to 10⁹ cells/individual per administration, and it is administered, for example, intravenously to a patient once or several times.

### Example 1

Production of alloreactive regulatory T cells by co-culturing dendritic cells induced from allo-derived iPS cells and regulatory T cells Materials:

iPS cells: iPS cells established from peripheral blood of a healthy volunteer (healthy volunteer A) in Institute for Frontier Life and Medical Sciences, Field of Regenerative Immunology, Kyoto University (Kyoto, Japan) were used.

Regulatory T cells (Treg): CD25-positive CD45RA-positive fraction of the cells isolated from peripheral blood of a healthy volunteer (healthy volunteer B) using FACSAria in Institute for Frontier Life and Medical Sciences, Field of Regenerative Immunology, Kyoto University (Kyoto, Japan) were used.

Monocytes: CD14-positive fraction of the cells isolated from peripheral blood of a healthy volunteer (healthy volunteer C) using MACS beads in Institute for Frontier Life and Medical Sciences, Field of Regenerative Immunology, Kyoto University (Kyoto, Japan) were used.

1) Differentiation of iPS Cells into Dendritic Cells Via Monocytes

Culture media used here are shown below.

TABLE 1
Medium A (for maintaining OP9 stromal cell)

| | | |
|---|---|---|
| | | Final |
| | | Concentration |
| Ingredients | Content | in the Medium |

| | | | |
|---|---|---|---|
| αMEM Medium | 500 | mL | |
| FCS | 125 | mL | 20% |
| Penicillin/ | 6.25 | mL | 1% |
| streptomycin | | | |
| Solution* | | | |
| Total | 631.25 | mL | |

*The penicillin/streptomycin solution had a composition of 10000 U/mL of penicillin and 10000 μg/mL of streptomycin, and hence the final concentrations thereof were respectively 100 U/mL and 100 μg/mL.

TABLE 2
Medium B (for induction of differentiation into monocyte)

| | | |
|---|---|---|
| | | Final |
| | | Concentration |
| Ingredients | Content | in Medium |

| αMEM Medium | 500 | mL | | |
|---|---|---|---|---|
| FCS | 125 | mL | 20% | |
| Penicillin/ | 5 | mL | 1% | |
| streptomycin | | | | |
| Solution* | | | | |
| hrGM-CSF | 630 | μL | 100 | ng/mL |
| (stock: 100 μg/ml) | | | | |
| hrM-CSF | 315 | μL | 50 | ng/mL |
| (stock: 100 μg/ml) | | | | |
| Total | 630.945 | mL | | |

*The penicillin/streptomycin solution had a composition of 10000 U/mL of penicillin and 10000 μg/mL of streptomycin, and hence the final concentrations thereof were respectively 100 U/mL and 100 μg/mL.

TABLE 3
Medium C (for induction of differentiation
into immature dendritic cell)

| | | |
|---|---|---|
| | | Final |
| | | Concentration |
| Ingredients | Content | in Medium |

| RPMI1640 | 500 | mL | | |
|---|---|---|---|---|
| FCS | 55 | mL | 10% | |
| 100x Non-Essential | 5 | mL | | |
| Amino Acids | | | | |
| Penicillin/ | 5 | mL | 1% | |
| streptomycin | | | | |
| Solution* | | | | |
| 2-Mercaptoethanol | 1.75 | μL | 0.05 | mM |
| hrGM-CSF | 126 | μL | 20 | ng/mL |
| (stock: 100 μg/ml) | | | | |
| hrIL-4 | 126 | μL | 20 | ng/mL |
| (stock: 100 μg/ml) | | | | |

| | |
|---|---|
| Total | 565.2537 |

*The penicillin/streptomycin solution had a composition of 10000 U/mL of penicillin and 10000 μg/mL of streptomycin, and hence the final concentrations thereof were respectively 100 U/mL and 100 μg/mL.

TABLE 4
Medium D (for induction of differentiation
into mature dendritic cell)

| | | |
|---|---|---|
| | | Final |
| | | Concentration |
| Ingredients | Content | in Medium |

| RPMI1640 | 500 | mL | | |
|---|---|---|---|---|
| FCS | 55 | mL | 10% | |
| 100x Non-Essential | 5 | mL | | |
| Amino Acids | | | | |
| Penicillin/ | 5 | mL | 1% | |
| streptomycin | | | | |
| Solution* | | | | |
| 2-Mercaptoethanol | 1.75 | μL | 0.05 | mM |
| hrIL-1β | 56.5 | μL | 10 | ng/mL |
| (stock: 100 μg/ml) | | | | |
| hrIL-6 | 56.5 | μL | 10 | ng/mL |
| (stock: 100 μg/ml) | | | | |
| hrTNFα | 56.5 | μL | 10 | ng/mL |
| (stock: 100 μg/ml) | | | | |
| hPGE2 | 56.5 | μL | 1 | ng/mL |
| (stock: 10 mg/ml) | | | | |
| Total | 565.2278 | mL | | |

*The penicillin/streptomycin solution had a composition of 10000 U/mL of penicillin and 10000 μg/mL of streptomycin, and hence the final concentrations thereof were respectively 100 U/mL and 100 μg/mL.

A. Preparation of OP9 Cells

Six milliliters (6 mL) of 0.1% gelatin solution in PBS was added to a 10 cm dish (Falcon) and incubated for 30 minutes at 37° C. OP9 stromal cells were detached from a confluent culture dish with trypsin/EDTA solution and about ¼ of the obtained cells were added to the gelatin coated 10 cm cell culture dish. 10 mL of medium A was added to the cell culture dish. Four days after, medium A 10 mL was added to the dish (final amount was 20 mL).

B. Induction of hematopoietic progenitor cells from iPS cells

Day 0: Seeding of the iPS Cells

The medium in the OP9 stromal cell culture to be used for the co-culture was aspirated and replaced with fresh medium A. The medium in the human iPS cell culture dish was also aspirated and 10 mL of fresh medium A was added. The iPS cell masses were removed from the bottom of the dish by using a dissociation medium and mechanically fragmented to smaller sizes by means of pipetting. The iPS cell clusters were suspended by means of pipetting. The number of the iPS cell clusters was visually counted and approximately 600 IFS cell clusters were seeded on the OP9 cells.

Two or more dishes per clone of iPS cells were used, and when subculturing, the cells in all dishes were once pooled in one dish and then redistributed to the same number of dishes to reduce the disparity between the dishes.

Day 1: (the Medium was Replaced)

It was confirmed that the iPS cell masses adhered to the dish and started to differentiate. The cell culture medium was replaced with 20 mL of fresh medium A.

Day 5: (a Half of the Medium was Replaced)

A half of the cell culture medium was replaced with 10 mL of fresh medium A.

Day 9: (a Half of the Medium was Replaced)

A half of the cell culture medium was replaced with 10 mL of fresh medium A.

Day 13: (Induced Mesodermal Cells were Transferred from

OP9 cell layer onto OP9/DLL1 cell layer)

Cell culture medium was aspirated and the surface of the cultured cells were washed with HESS(⁺M⁺Ca) to washout the cell culture medium. 10 mL of Collagenase IV 250 U in HBSS (+Mg+Ca) solution was added to the dish and incubated for 45 minutes at 37° C.

The collagenase solution was removed by aspiration and the cells were washed with 10 mL of PBS(−). Then, 0.05% trypsin/EDTA solution was added to the dish and the dish was incubated for 20 minutes at 37° C. After the incubation, the sheet like cell aggregates peeled from the bottom of the dish and the cell aggregates were mechanically fragmented to smaller sizes by means of pipetting. Thus treated cells were added with fresh medium A 20 mL and cultured for more 45 minutes at 37° C. The culture medium containing the floating cells was passed through 100 μm mesh and the cells were collected. The cells were then centrifuged at 1200 rpm for 7 minutes at 4° C. The obtained pellet was suspended in 10 mL of medium B. One-tenth of the suspension was separated and used for the FACS analysis. The remaining cell suspension was seeded to new dishes containing OP9/DLL1 cells. Cell suspensions obtained from several dishes were pooled and the pooled cells were seeded to the same number of new dishes.

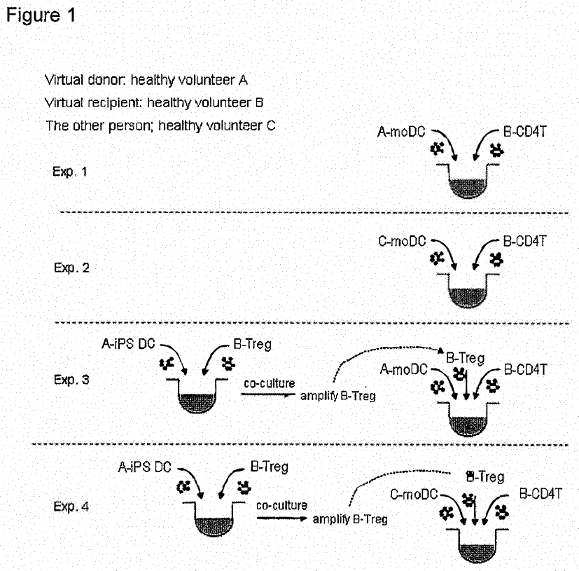

In order to ascertain whether or not hematopoietic progenitor cells were contained in the obtained cells, FACS analysis was carried out using anti-CD34 antibody, anti-CD43 antibody. A sufficient number of cells could be confirmed in the CD34^{low}CD43⁺ cell fraction and therefore, hematopoietic progenitor cells were induced. (FIG. 1)

C. Induction of Differentiation of Hematopoietic Progenitor Cells into Monocytes

Subsequently, all the cultured cells containing the CD34^{low}CD43⁺ cell fraction were seeded in a 10 cm cell culture dish.

During the culturing period, FACS analysis was conducted several times to confirm the differentiation stages. A considerable number of dead cells were observed over the culturing period. Dead cells were preferably eliminated by using, for example, propidium Iodide (PI) or 7-AAD before the FACS analysis.

Day 14 (Cells were Subcultured)

The suspended cells were collected into a 50 mL conical tube through a 100 μm mesh screen after gently pipetting several times. The thus collected cells were centrifuged at 4° C. and 1200 rpm for 7 minutes, and thus obtained pellet was suspended in 10 mL of medium B. The resultant cells were seeded in a 10 cm cell culture dish separately prepared.

Day 18 (Culture Medium was Replaced)

All the cells were collected into a 50 mL conical tube through a 100 μm mesh screen after gently pipetting several times. The thus collected cells were centrifuged at 4° C. and 1200 rpm for 7 minutes, and the thus obtained pellet was suspended in 10 mL of medium B. The resultant cells were seeded in a 10 cm cell culture dish separately prepared.

D. Differentiation of monocytes into dendritic cells Day 21: Generation of monocytes (CD14⁺ cells) were confirmed. Those cells were differentiated into immature dendritic cells. The cells were dispersed by gentle pipetting and all cells were collected into a 50 mL conical tube through a 100 μm mesh screen. The number of the cells was counted and the cells were centrifuged at 4° C. and 1200 rpm for 7 minutes. The obtained pellet was suspended in medium C. The number of the cells was adjusted to 5×10³ cells/ml, and the cell suspension was seeded in a 24-well plate at 1 mL/well.

Day 23 (Culture Medium was Replaced)

The cells were dispersed by pipetting gently and all cells were collected into a 50 mL conical tube through a 100 μm mesh screen. The thus collected cells were centrifuged at 4° C. and 1200 rpm for 7 minutes, the obtained pellet was suspended in medium C, and the resultant cells were seeded again in a 24-well plate.

Day 25 (Culture Medium was Replaced)

The cells were dispersed by pipetting gently and all cells were collected into a 50 mL conical tube through a 100 μm mesh screen. The thus collected cells were centrifuged at 4° C. and 1200 rpm for 7 minutes, the thus obtained pellet was suspended in medium C, and the resultant cells were seeded again in a 24-well plate.

Day 27: Generation of Immature Dendritic Cells was Confirmed

It was visually confirmed that immature dendritic cells had been produced. Differentiation of the immature cells into mature dendritic cells was started. The cells were dispersed by gentle pipetting and all cells were collected into a 50 mL conical tube through a 100 μm mesh screen. Thus collected cells were centrifuged at 4° C. and 1200 rpm for 7 minutes, the thus obtained pellet was suspended in medium D, and the resultant cell suspension was seeded again in a 24-well plate.

Day 28: Generation of Mature Dendritic Cells was Confirmed.

It was visually confirmed that mature dendritic cells had been produced. All the cells were collected and washed twice with RPMI1640/10% FCS medium. Then, the cells were used in the following experiments.

2) Selective Amplification of the Alloreactive Regulatory T Cells

A population of CD4⁺CD45RA⁺CD25^{high} cells was isolated from peripheral blood mononuclear cells (PBMC) of the healthy volunteer B using a flow cytometer. The cells were used as a population of regulatory T cells.

Mature dendritic cells derived from iPS cells established from the healthy volunteer A (A-derived mature dendritic cell) and the regulatory T cells isolated from the healthy volunteer B (regulatory T cell of B) were co-cultured.

A U-bottom 96-well plate was used. A-derived mature dendritic cells and the regulatory T cells of B were added together to each well in amounts of 1.0×10⁴ cells and 1.0×10⁴ cells respectively to give dendritic cell: regulatory T cell ratio of 10:1.

The cells thus mixed were cultured in a culture medium supplemented with 20 U/ml IL-2 at 5% CO₂ and 37° C. for another 2 weeks.

The regulatory T cells were proliferated 30 to 50 fold in the 2 weeks co-culture.

It has been known that regulatory T cells are activated when IL-2 is present in the culture medium. In order to eliminate the influence of IL-2 on the behavior of the regulatory T cells, the culture medium was replaced with a medium containing no IL-2 on one day before using the cells in the following example wherein the inhibiting ability of the cells were evaluated. The cells were cultured for one day in the absence of IL-2 before being used in the example.

3) Evaluation of the Alloreactive Regulatory T Cells to Inhibit the Immune Reaction

The following test was designed on the assumption of:

Virtual donor: healthy volunteer A

Virtual recipient: healthy volunteer B

The other person: healthy volunteer C. The outline of this experiment is illustrated in FIG. 1.

The regulatory T cells alloreactive to the healthy volunteer A, that were induced from the regulatory T cells derived from the healthy volunteer B obtained as described in 2) above were used. In order to evaluate the effect to inhibit the immune reaction, a mixed lymphocyte reaction assay using the alloreactive T cells was performed.

Cells in the CD4⁺CD45RA⁺CD25^{nega} fraction isolated from the peripheral blood of the healthy volunteer B, i.e. a cell population that does not contain regulatory T cells were used as responder cells. The cells in the fraction were labelled with CellTrace Violet (CTV). The regulatory T cells that were alloreactive to the healthy volunteer A and were derived from the healthy volunteer B obtained in 2) above were labeled with CFSE.

Mature dendritic cells induced from monocytes contained in the peripheral blood of the healthy volunteer A and mature dendritic cells induced from monocytes in the peripheral blood of the healthy volunteer C were used as stimulator cells.

A U-bottom 96-well plate was used. The responder cells, stimulator cells, and alloreactive regulatory T cells were added together to each well in amounts of 1.0×10⁴ cells, 1.0×10⁴ cells and 0.66×10⁴ cells per well respectively. As a control group, the responder cells and stimulator cells but not the alloreactive regulatory T cells were added to the well. The outline of this experiment is illustrated in FIG. 1.

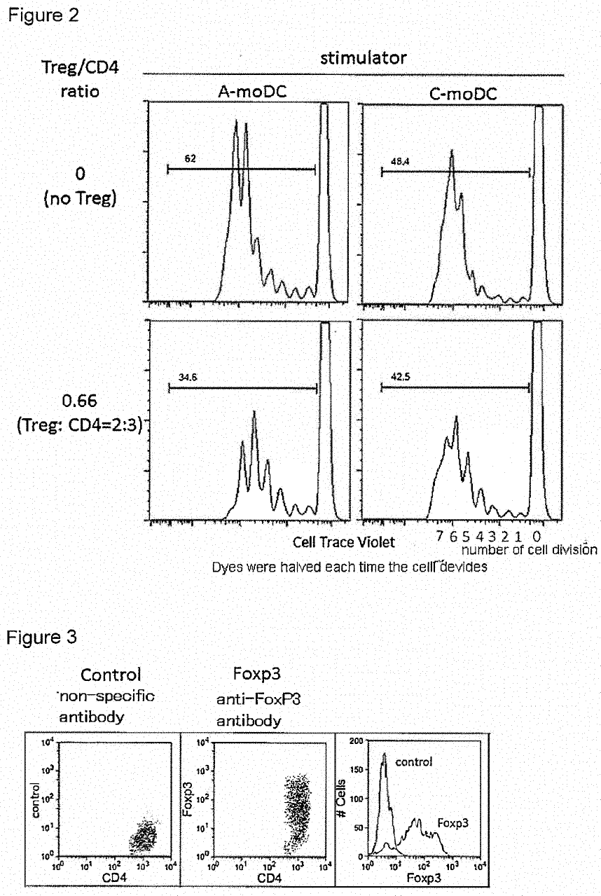

The mixture of the cells was cultured for 4 days, and the number of the responder cells was analyzed using a flow cytometer and the degree of proliferate was evaluated. Specifically, the analysis was performed by using, as an index, the intensity of CTV in the CD4-positive responder cells contained in the fraction not containing the alloreactive regulatory T cells (CFSE⁻ fraction). The cell growth rate of the control group was taken as 100% and the inhibition effect attained by adding the alloreactive regulatory T cells was calculated. The results are illustrated in FIG. 2.

Experiments 1 and 2 correspond to controls, and it was found that CD4 T cells were proliferated in response to the dendritic cells derived from the monocytes of the healthy volunteer A and the dendritic cells derived from the monocytes of the healthy volunteer C, both added as the stimulators. The cells proliferated by 62.0% and 48.4%, respectively.

Experiment 3 corresponds to the present method, and it was confirmed that the proliferation of the CD4 T cells was inhibited when the regulatory T cells obtained by co-culturing the dendritic cells derived from iPS cells produced from the peripheral blood of the volunteer A and the regulatory T cells obtained from the volunteer B were added to a system in which the dendritic cells derived from the monocytes of the volunteer A were used as stimulator. The cells proliferated by 34.6%. When the cell proliferation in Experiment 1 (control) is taken as 100%, the proliferation of the cells is 56.0%.

In Experiment 4, the regulatory T cells obtained by co-culturing the dendritic cells derived from iPS cells produced from the peripheral blood of the volunteer A and the regulatory T cells obtained from the volunteer B were added to a system in which the dendritic cells derived from the monocytes of the volunteer C were used as the stimulator. The regulatory T cells added were expected to be specific to the volunteer A, and were presumed not to inhibit the proliferation of the CD4 T cells that do not contain the regulatory T cell derived from the person B in response to the stimulator derived from the monocytes of the volunteer C.

In Experiment 4, the cells proliferated by 42.5%. When the cell proliferation in Experiment 2 (control) is taken as 100%, the proliferation of the cells is 88.0%. Thus, as compared with Experiment 3, the influence of the regulatory T cells on the cell proliferation was very low.

### Example 2

Production of alloreactive regulatory T cells by co-culturing dendritic cells induced from HLA-homo iPS cells and regulatory T cells

Materials

HLA-homo iPS cells (D): iPS cells established from somatic cells of a person a homozygous HLA class II haplotype at Center for iPS Cell Research and Application, Kyoto University were used.

Regulatory T cells (Treg): CD25-positive CD45RA-positive fraction of the cells isolated from peripheral blood of a healthy volunteer (healthy volunteer E) using FACSAria in Institute for Frontier Life and Medical Sciences, Field of Regenerative Immunology, Kyoto University (Kyoto, Japan) were used.

HLA haplotypes of HLA homo-iPS cells (D) do not match any of the HLA haplotypes of healthy volunteer E.

1) Differentiation of iPS Cells into Dendritic Cells Via Monocytes

Dendritic cells were obtained from iPS cells in the same manner as Example 1.

2) Selective Amplification of the Alloreactive Regulatory T Cells

A population of CD4÷CD45RA⁺CD25^{high} cells was isolated from peripheral blood mononuclear cells (PBMC) of the healthy volunteer E using a flow cytometer. The cells were used as a population of regulatory T cells.

Mature dendritic cells derived from iPS cells established from the healthy volunteer E (E-derived mature dendritic cell) and the regulatory T cells isolated from the healthy volunteer E (regulatory T cell of E) were co-cultured.

A U-bottom 96-well plate was used. A-derived mature dendritic cells and the regulatory T cells of D were added together to each well in amounts of 1.0×10⁴ cells and 1.0×10⁴ cells respectively to give dendritic cell: regulatory T cell ratio of 10:1.

The cells thus mixed were cultured in a culture medium supplemented with 20 U/ml IL-2 at 5% CO₂ and 37° C. for another two weeks.

The regulatory T cells were proliferated 30 to 50 fold in the 2 weeks co-culture. After that, the regulatory T cells were co-cultured with anti-CD3/CD28 beads at 5% CO₂ and 37° C. for another one week. The medium used here was the same that used for the co-culturing with dendritic cells.

Expression of Foxp3 on the obtained cells were evaluated. Result is shown in FIG. 3. The amplified alto-antigen-specific regulatory T cells stably expressed Foxp3.

When the culture medium contained rapamycin, proliferation of CD4 positive T cells other than Foxp3 expression regulatory T cells were duly suppressed.

### EXPRESSION OF REFERENCE LETTERS

moDC: mature dendritic cells derived from monocytes, CD4 T: CD4-positive T cells not containing regulatory T cell, iPS DC: mature dendritic cells derived from iPS cells, Treg: regulatory T cells.

What is claimed is:

1. A method for producing regulatory T cells for inducing immune tolerance in a subject, comprising the step of co-culturing regulatory T cells obtained from the subject with dendritic cells derived from iPS cells.

2. The method according to claim 1, comprising the steps of:
providing iPS cells established from a somatic cell of a somatic cell donor having HLA class II molecules that match with those of the subject to a certain extent or more;
inducing dendritic cells from the iPS cells;
sensitizing the dendritic cells with an antigen to which the immune tolerance is to be induced to give antigen-presenting dendritic cells; and
co-culturing regulatory T cells obtained from the subject with the antigen-presenting dendritic cells.

3. The method according to claim 1, wherein the subject is a transplant recipient, and immune tolerance against a transplant donor is induced, comprising the steps of:
providing iPS cells established from a somatic cell derived from a somatic cell donor having HLA class II molecules that match with those of the transplant donor to a certain extent or more;
inducing dendritic cells from the iPS cells; and
co-culturing regulatory T cells obtained from the transplant recipient with the induced dendritic cells.

4. The method according to claim 1, wherein the subject is a transplant recipient, and immune tolerance against a transplant donor is induced, comprising the steps of:
providing iPS cells established from a somatic cell derived from a somatic cell donor having HLA class II molecules that match with those of the transplant recipient to a certain extent or more;
inducing dendritic cells from the iPS cells;
sensitizing the dendritic cells with an antigen derived from the transplant donor to give antigen-presenting dendritic cells; and
co-culturing regulatory T cells obtained from the transplant recipient with the induced antigen-presenting dendritic cells.

5. A method for inducing antigen-specific regulatory T cells for tolerating the immune reaction caused by the T cells in a tissue of a transplant donor against a transplant recipient, comprising the steps of:
preparing iPS cells established from a somatic cell derived from a somatic cell donor having HLA class II molecules that match that with those of the transplant recipient to a certain extent or more;
inducing dendritic cells from the iPS cells; and
co-culturing regulatory T cells obtained from the transplant donor with the induced dendritic cells.

6. A method for inducing antigen-specific regulatory T cells for tolerating the immune reaction caused by the T cells in a tissue of a transplant donor against a transplant recipient, comprising the steps of:
providing iPS cells established from a somatic cell derived from a somatic cell donor having HLA class II molecules that match with those of the transplant donor to a certain extent or more;
inducing dendritic cells from the iPS cells;
sensitizing the dendritic cells with an antigen derived from the transplant recipient; and
co-culturing regulatory T cells obtained from the transplant donor with the antigen-presenting dendritic cells.

7. The method according to claim 3, wherein the step of sensitizing the dendritic cells with the antigen comprises a step of causing the dendritic cells to incorporate a protein derived from the cells of the organ to which the immune tolerance is to be introduced.

8. The method according to any of claim 1, wherein the ratio between the dendritic cells and the regulatory T cells is 1:1 to 20:1 at the beginning of co-culturing the regulatory T cells with the dendritic cells.

9. The method according to claim 1, wherein the regulatory T cells and the dendritic cells are co-cultured for about 1 to 2 weeks.

10. The method according to claim 1, wherein the regulatory T cells and the dendritic cells are co-cultured in the presence of rapamycin.

11. The method according to claim 6, wherein the step of sensitizing the dendritic cells with the antigen comprises a step of causing the dendritic cell to incorporate a protein derived from the cells of the organ to which the immune tolerance is to be introduced.

12. The method according to claim 5, wherein the ratio between the dendritic cells and the regulatory T cells is 1:1 to 20:1 at the beginning of co-culturing the regulatory T cells with the dendritic cells.

13. The method according to claim 6, wherein the ratio between the dendritic cells and the regulatory T cells is 1:1 to 20:1 at the beginning of co-culturing the regulatory T cells with the dendritic cells.

14. The method according to claim 5, wherein the regulatory T cells and the dendritic cells are co-cultured for about 1 to 2 weeks.

15. The method according to claim 6, wherein the regulatory T cells and the dendritic cells are co-cultured for about 1 to 2 weeks.

16. The method according to claim 5, wherein the regulatory T cells and the dendritic cells are co-cultured in the presence of rapamycin.

17. The method according to claim 6, wherein the regulatory T cells and the dendritic cells are co-cultured in the presence of rapamycin.
